(19) United States
(12) Patent Application Publication
(10) Pub. No.: US 2023/0290473 A1
(43) Pub. Date: Sep. 14, 2023

(54) System, Method, and Computer Program Product for Medication Administration

(71) Applicant: Becton, Dickinson and Company, Franklin Lakes, NJ (US)

(72) Inventors: Ashley Rachel Rothenberg, Morris Plains, NJ (US); Erik Kurt Witt, Oakland, NJ (US); Mark Jesser, Wyckoff, NJ (US); Ami Kumordzie, New York, NY (US)

(21) Appl. No.: 18/008,380

(22) PCT Filed: Jun. 3, 2021
(86) PCT No.: PCT/US2021/035650

§ 371 Date: Dec. 5, 2022

Related U.S. Application Data
(60) Provisional application No. 63/034,755, filed on Jun. 4, 2020.

(51) Int. Cl. G16H 20/17; G16H 70/40

(52) U.S. Cl. CPC G16H 20/17; G16H 70/40

(57) ABSTRACT

A method may include obtaining pathway data associated with a medication delivery pathway for delivering medication to a patient, the medication delivery pathway including at least one of the following: at least one catheter, at least one lumen, at least one port, or any combination thereof; obtaining prescription data associated with (i) a medication prescribed for delivery to the patient and (ii) another medication delivered, being delivered, or prescribed to be delivered to the patient; determining, according to a rule set, based on the pathway data and the prescription data, a medication delivery pathway for delivery of the medication to the patient; and assigning the medication for delivery to the patient via the medication delivery pathway.

### CROSS-REFERENCE TO RELATED APPLICATIONS

This application is the United States national phase of International Application No. PCT/US2021/035650 filed Jun. 3, 2021, and claims priority to U.S. Provisional Application Ser. No. 63/034,755 filed Jun. 4, 2020, entitled System, Method, and Computer Program Product for Medication Administration, the entire disclosures of which are hereby incorporated by reference in their entirety.

### BACKGROUND

### 1. Field

This disclosure relates generally to medication administration, and in some non-limiting embodiments or aspects, to a system, a method, and a product for determining a medication delivery pathways for delivery of medication to a patient.

### 2. Technical Considerations

Various existing systems provide information associated with medication compatibility. However, a health care practitioner may be required to access these systems to determine the compatibility of various medications prescribed to a patient, and these systems do not provide the health care practitioner with information on available medication delivery pathways for delivering medication to the patient and/or what medications have been delivered and/or are currently being delivered through which medication delivery pathways for the patient.

### SUMMARY

Accordingly, provided are improved systems, methods, and/or products for medication administration.

According to some non-limiting embodiments or aspects, provided is a computer-implemented method, including: obtaining, with at least one processor, pathway data associated with at least one medication delivery pathway for delivering medication to a patient, wherein the at least one medication delivery pathway includes at least one of the following: at least one catheter, at least one lumen, at least one port, or any combination thereof; obtaining, with at least one processor, prescription data associated with (i) one or more medications prescribed for delivery to the patient and (ii) at least one other medication delivered, being delivered, or prescribed to be delivered to the patient; determining, with at least one processor, according to at least one rule set, based on the pathway data and the prescription data, one or more medication delivery pathways for delivery of the one or more medications to the patient; and assigning, with at least one processor, the one or more medications for delivery to the patient via the one or more medication delivery pathways.

According to some non-limiting embodiments or aspects, provided is a system including: one or more processors programmed and/or configured to: obtain pathway data associated with at least one medication delivery pathway for delivering medication to a patient, wherein the at least one medication delivery pathway includes at least one of the following: at least one catheter, at least one lumen, at least one port, or any combination thereof; obtain prescription data associated with (i) one or more medications prescribed for delivery to the patient and (ii) at least one other medication delivered, being delivered, or prescribed to be delivered to the patient; determine according to at least one rule set, based on the pathway data and the prescription data, one or more medication delivery pathways for delivery of the one or more medications to the patient; and assign the one or more medications for delivery to the patient via the one or more medication delivery pathways.

According to some non-limiting embodiments or aspects, provided is a computer program product including at least one non-transitory computer-readable medium including program instructions that, when executed by at least one processor, cause the at least one processor to: obtain pathway data associated with at least one medication delivery pathway for delivering medication to a patient, wherein the at least one medication delivery pathway includes at least one of the following: at least one catheter, at least one lumen, at least one port, or any combination thereof; obtain prescription data associated with (i) one or more medications prescribed for delivery to the patient and (ii) at least one other medication delivered, being delivered, or prescribed to be delivered to the patient; determine according to at least one rule set, based on the pathway data and the prescription data, one or more medication delivery pathways for delivery of the one or more medications to the patient; and assign the one or more medications for delivery to the patient via the one or more medication delivery pathways.

Further non-limiting embodiments or aspects are set forth in the following numbered clauses:

Clause 1. A computer-implemented method, comprising: obtaining, with at least one processor, pathway data associated with at least one medication delivery pathway for delivering medication to a patient, wherein the at least one medication delivery pathway includes at least one of the following: at least one catheter, at least one lumen, at least one port, or any combination thereof; obtaining, with at least one processor, prescription data associated with (i) one or more medications prescribed for delivery to the patient and (ii) at least one other medication delivered, being delivered, or prescribed to be delivered to the patient; determining, with at least one processor, according to at least one rule set, based on the pathway data and the prescription data, one or more medication delivery pathways for delivery of the one or more medications to the patient; and assigning, with at least one processor, the one or more medications for delivery to the patient via the one or more medication delivery pathways.

Clause 2. The computer-implemented method of clause 1, wherein determining the one or more medication delivery pathways includes: determining, according to the at least one rule set, based on the pathway data and the prescription data, whether the one or more medications are compatible to be delivered to the patient via a same medication delivery pathway as the at least one medication based on the pathway data and the prescription data.

Clause 3. The computer-implemented method of any of clauses 1 and 2, wherein determining whether the one or more medications are compatible to be delivered to the patient via the same medication delivery pathway as the at least one medication includes: determining, according to the at least one rule set, based on the pathway data and the prescription data, whether the one or more medications are compatible to be delivered to the patient via the same medication delivery pathway as the at least one medication if the same medication delivery pathway is flushed in between deliveries of the one or more medications and the at least one medication.

Clause 4. The computer-implemented method of any of clauses 1-3, wherein the one or more medication delivery pathways include the at least one medication delivery pathway, and wherein assigning the one or more medications for delivery to the patient via the one or more medication delivery pathways includes: prompting a user to flush the one or more medication delivery pathways at least one of before and after delivering the one or more medications via the one or more medication delivery pathways.

Clause 5. The computer-implemented method of any of clauses 1-4, wherein assigning the one or more medications for delivery to the patient via the one or more medication delivery pathways includes: prompting a user to install the one or more medication delivery pathways at a body of the patient.

Clause 6. The computer-implemented method of any of clauses 1-5, further comprising: segmenting, with at least one processor, according to the at least one rule set, the one or more medications and the at least one medication into a plurality of medication groups based on whether the one or more medications and the at least one medication are compatible to be delivered to the patient via the same medication delivery pathway; and determining, with at least one processor, based on the pathway data and the prescription data, according to the at least one rule set, the one or more medication delivery pathways for delivery of the plurality of medication groups to the patient based on the pathway data and the prescription data.

Clause 7. The computer-implemented method of any of clauses 1-6, wherein determining whether the one or more medications are compatible to be delivered to the patient via the same medication delivery pathway as the at least one medication includes: determining, according to the at least one rule set, based on the pathway data and the prescription data, whether the one or more medications are compatible to be delivered to the patient via a same catheter as the at least one medication.

Clause 8. The computer-implemented method of any of clauses 1-7, wherein determining whether the one or more medications are compatible to be delivered to the patient via the same medication delivery pathway as the at least one medication includes: determining, according to the at least one rule set, based on the pathway data and the prescription data, whether the one or more medications are compatible to be delivered to the patient via a same lumen of the same catheter as the at least one medication.

Clause 9. The computer-implemented method of any of clauses 1-8, wherein determining whether the one or more medications are compatible to be delivered to the patient via the same medication delivery pathway as the at least one medication includes: determining, according to the at least one rule set, based on the pathway data and the prescription data, whether the one or more medications are compatible to be delivered to the patient via a same port of the same catheter as the at least one medication.

Clause 10. The computer-implemented method of any of clauses 1-9, wherein the pathway data is associated with at least one type of the at least one medication delivery pathway, and wherein determining the one or more medication delivery pathways includes: determining, according to the at least one rule set, based on the pathway data and the prescription data, whether the one or more medications are compatible to be delivered to the patient via the one or more types of the at least one medication delivery pathway.

Clause 11. The computer-implemented method of any of clauses 1-10, wherein the one or more types of the at least one medication delivery pathway include at least one of the following: a peripheral intravenous line (PIV), a peripherally inserted central catheter (PICC), a central catheter, a midline catheter, or any combination thereof.

Clause 12. The computer-implemented method of any of clauses 1-11, wherein the pathway data is associated with at least one location of a body of the patient to which the at least one medication delivery pathway is configured to deliver medication, and wherein determining the one or more medication delivery pathways includes: determining, according to the at least one rule set, based on the pathway data and the prescription data, whether the one or more medications are compatible to be delivered to the patient at the one or more locations of the body of the patient.

Clause 13. The computer-implemented method of any of clauses 1-12, wherein the pathway data is associated with at least one material of the at least one medication delivery pathway, and wherein determining the one or more medication delivery pathways includes: determining, according to the at least one rule set, based on the pathway data and the prescription data, whether the one or more medications are compatible to be delivered to the patient via a pathway including the one or more materials.

Clause 14. The computer-implemented method of any of clauses 1-13, wherein determining whether the one or more medications are compatible to be delivered to the patient via the at least one medication delivery pathway including the one or more materials includes: determining, according to the at least one rule set, based on the pathway data and the prescription data, an amount of time the one or more medications are compatible to be delivered to the patient via the pathway including the one or more materials.

Clause 15. The computer-implemented method of any of clauses 1-14, wherein the prescription data is associated with one or more delivery types of the one or more medications, one or more dosage amounts of the one or more medications, one or more dosage rates of the one or more medications, one or more time periods acceptable for delivering the one or more medications to the patient, or any combination thereof.

Clause 16. The computer-implemented method of any of clauses 1-15, wherein determining the one or more medication delivery pathways includes: determining, according to the at least one rule set, based on the pathway data and the prescription data, one or more first time periods for delivery of the one or more medications via the one or more medication delivery pathways.

Clause 17. The computer-implemented method of any of clauses 1-16, wherein determining the one or more medication delivery pathways includes: determining, according to the at least one rule set, based on the pathway data and the prescription data, one or more second time periods after the one or more first time periods for delivery of one or more second doses of the one or more medications via the one or more medication delivery pathways.

Clause 18. The computer-implemented method of any of clauses 1-17, wherein the at least one rule set includes a priority of delivery for the one or more medications and the at least one medication.

Clause 19. The computer-implemented method of any of clauses 1-18, wherein the at least one rule set includes a priority of delivery of for the one or more medications via a type, material, and/or a location of medication delivery pathway.

Clause 20. The computer-implemented method of any of clauses 1-19, wherein the at least one other medication was delivered or is being delivered via the at least one medication delivery pathway, wherein the pathway data is received from at least one sensor coupled to the at least one medication delivery pathway, and wherein the pathway data is associated with the at least one other medication delivered or being delivered via the at least one medication delivery pathway.

Clause 21. A system comprising: one or more processors programmed and/or configured to: obtain pathway data associated with at least one medication delivery pathway for delivering medication to a patient, wherein the at least one medication delivery pathway includes at least one of the following: at least one catheter, at least one lumen, at least one port, or any combination thereof; obtain prescription data associated with (i) one or more medications prescribed for delivery to the patient and (ii) at least one other medication delivered, being delivered, or prescribed to be delivered to the patient; determine according to at least one rule set, based on the pathway data and the prescription data, one or more medication delivery pathways for delivery of the one or more medications to the patient; and assign the one or more medications for delivery to the patient via the one or more medication delivery pathways.

Clause 22. A computer program product comprising at least one non-transitory computer-readable medium including program instructions that, when executed by at least one processor, cause the at least one processor to: obtain pathway data associated with at least one medication delivery pathway for delivering medication to a patient, wherein the at least one medication delivery pathway includes at least one of the following: at least one catheter, at least one lumen, at least one port, or any combination thereof; obtain prescription data associated with (i) one or more medications prescribed for delivery to the patient and (ii) at least one other medication delivered, being delivered, or prescribed to be delivered to the patient; determine according to at least one rule set, based on the pathway data and the prescription data, one or more medication delivery pathways for delivery of the one or more medications to the patient; and assign the one or more medications for delivery to the patient via the one or more medication delivery pathways.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments or aspects that are illustrated in the accompanying schematic figures, in which:

FIG. 1 is a diagram of non-limiting embodiments or aspects of an environment in which systems, devices, products, apparatus, and/or methods, described herein, can be implemented;

FIG. 2 is a diagram of non-limiting embodiments or aspects of an implementation of one or more systems and/or one or more devices of FIG. 1;

FIG. 3 is a diagram of non-limiting embodiments or aspects of components of one or more devices and/or one or more systems of FIGS. 1 and 2;

FIG. 4 is a flowchart of non-limiting embodiments or aspects of a process for medication administration;

FIG. 5 is a flowchart of non-limiting embodiments or aspects of a process for medication administration; and

FIG. 6a is a flowchart of an implementation of non-limiting embodiments or aspects of a process for medication administration.

FIG. 6b is a flowchart of an implementation of non-limiting embodiments or aspects of a process for medication administration.

FIG. 6c is a flowchart of an implementation of non-limiting embodiments or aspects of a process for medication administration.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to non-limiting embodiments or aspects as they are oriented in the drawing figures. However, it is to be understood that non-limiting embodiments or aspects may assume various alternative variations and step sequences, except where expressly specified to the contrary. It is also to be understood that the specific devices and processes illustrated in the attached drawings, and described in the following specification, are simply exemplary embodiments or aspects. Hence, specific dimensions and other physical characteristics related to the embodiments or aspects of the embodiments or aspects disclosed herein are not to be considered as limiting unless otherwise indicated.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

As used herein, the term “server” may refer to or include one or more processors or computers, storage devices, or similar computer arrangements that are operated by or facilitate communication and processing for multiple parties in a network environment, such as the Internet, although it will be appreciated that communication may be facilitated over one or more public or private network environments and that various other arrangements are possible. Further, multiple computers, e.g., servers, or other computerized devices, such as POS devices, directly or indirectly communicating in the network environment may constitute a “system,” such as a merchant's POS system.

Non-limiting embodiments or aspects of the present disclosure are directed to systems, devices, products, apparatus, and/or methods.

In this way, non-limiting embodiments, of the present disclosure provide for reducing in-line medication incompatibility issues (e.g., precipitating of two incompatible medications that clog a line, etc.) through creation and/or use of a dynamic patient specific record of available medication delivery pathways for a patient and what medications have been delivered through which medication delivery pathways for the patient by evaluating and/or optimizing assignment of new medication orders or prescriptions to medication delivery pathways according to a rule set (e.g., according to patient specific history, hospital rules, medication rules, medication-medication compatibility rules, etc.), as well for as improving medication delivery pathway assignment to reduce future requirements for flushing, to reduce a future need to add more medication delivery pathways to the patient, and/or to reduce future incidences of medication incompatibility.

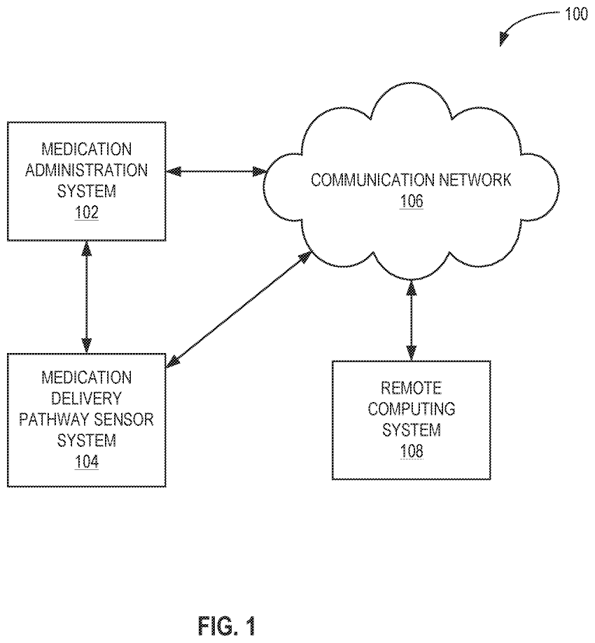

Referring now to FIG. 1, FIG. 1 is a diagram of an example environment 100 in which systems, devices, products, apparatus, and/or methods described herein, may be implemented. As shown in FIG. 1, environment 100 includes medication administration system 102, medication delivery pathway sensor system 104, communication network 106, and/or remote computing system 108. Systems and/or devices of environment 100 can interconnect via wired connections, wireless connections, or a combination of wired and wireless connections.

Medication administration system 102 may include one or more devices capable of receiving information and/or data from medication delivery pathway sensor system 104 and/or remote computing system 108 (e.g., via communication network 106, etc.) and/or communicating information and/or data to medication delivery pathway sensor system 104 and/or remote computing system 108 (e.g., via communication network 106, etc.). For example, medication administration system may include one or more computing systems including one or more processors (e.g., one or more computing devices, one or more mobile computing devices, one or more servers, etc.).

In some non-limiting embodiments or aspects, medication administration system 102 may be capable of receiving (e.g., from medication delivery pathway sensor system 104, etc.) information and/or data (e.g., pathway data, prescription data, etc.) via a short range wireless communication connection (e.g., an NFC communication connection, an RFID communication connection, a Bluetooth® communication connection, and/or the like), and/or communicating (e.g., to medication delivery pathway sensor system 104, etc.) information and/or data (e.g., pathway data, prescription data, etc.) via a short range wireless communication connection.

In some non-limiting embodiments or aspects, medication administration system 102 may include and/or access one or more internal and/or external databases including pathway data, prescription data, one or more rule sets including one or more rules (e.g., rule set data, etc.), and/or the like.

Medication delivery pathway sensor system 104 may include one or more devices capable of receiving information and/or data from medication administration system 102 and/or remote computing system 108 (e.g., via communication network 106, etc.) and/or communicating information and/or data to medication administration system 102 and/or remote computing system 108 (e.g., via communication network 106, etc.). For example, medication administration system may include one or more computing systems including one or more processors (e.g., one or more computing devices, one or more mobile computing devices, one or more servers, etc.).

In some non-limiting embodiments or aspects, medication delivery pathway sensor system 104 may be capable of receiving (e.g., from medication administration system 102, etc.) information and/or data (e.g., pathway data, prescription data, etc.) via a short range wireless communication connection (e.g., an NFC communication connection, an RFID communication connection, a Bluetooth® communication connection, and/or the like), and/or communicating (e.g., to medication administration system 102, etc.) information and/or data (e.g., pathway data, prescription data, etc.) via a short range wireless communication connection.

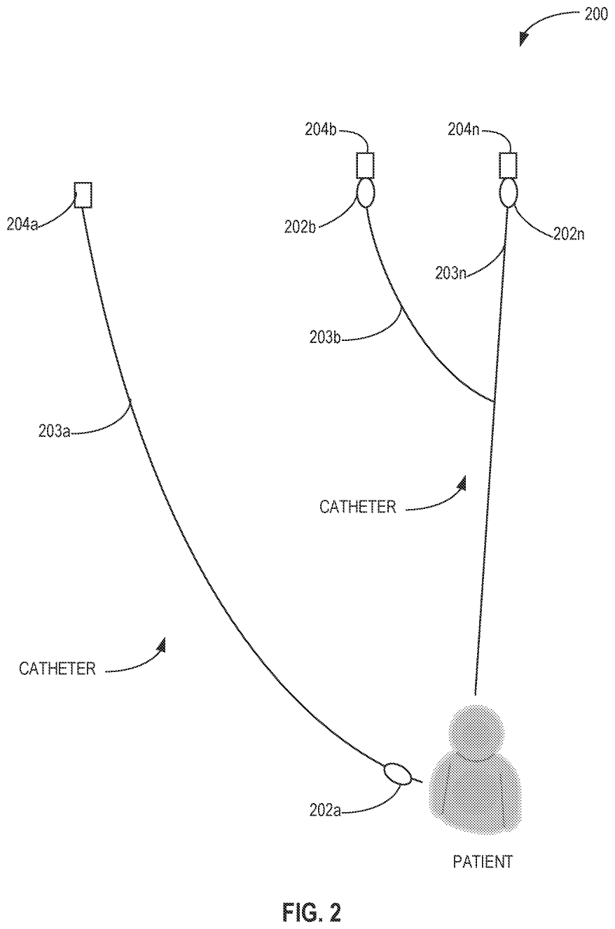

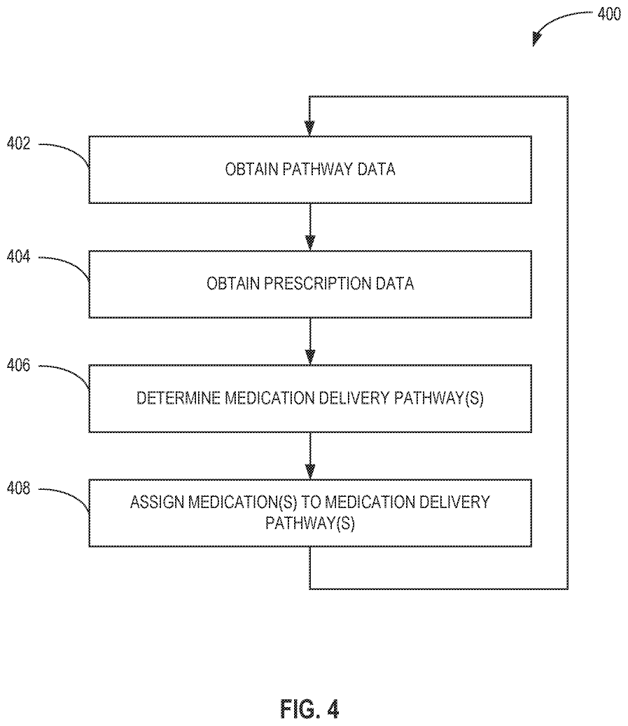

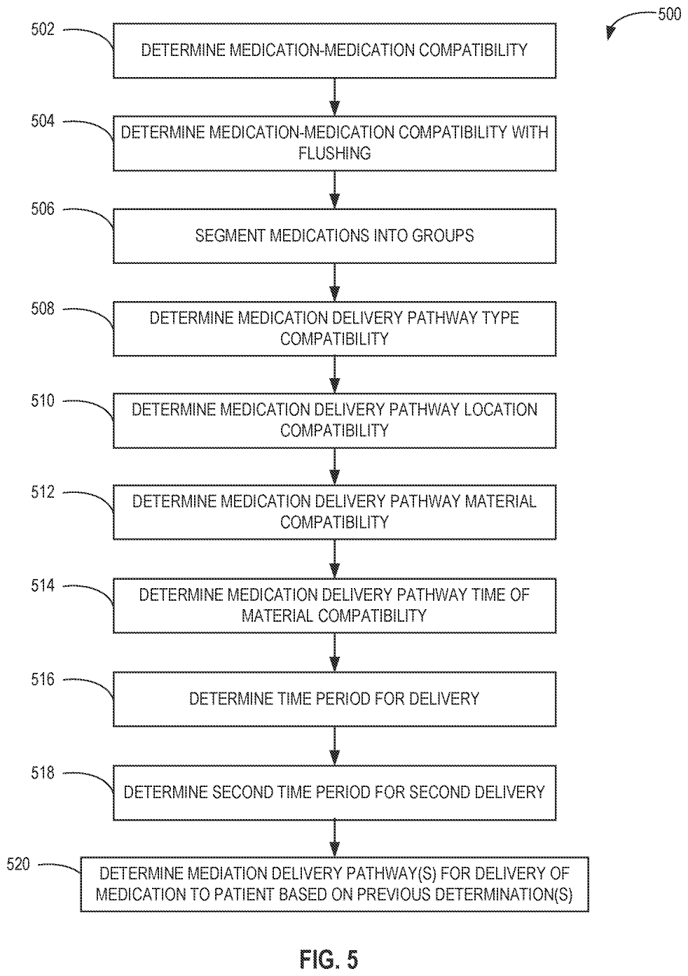

Referring also to FIG. 2, in some non-limiting embodiments or aspects, medication delivery pathway system 104 may include one or more sensors 202a, 202b, . . . 202n connected to one or more medication delivery pathways 203a, 203b, . . . 203n for delivering medication to a patient. For example, a medication delivery pathway may include at least one of the following: at least one catheter, at least one lumen, at least one port, or any combination thereof. As an example, the one or more medication delivery pathways 203a, 203b, . . . 203n may include one or more ports 204a, 204b, . . . 204n (e.g., fluid ports for receiving a fluid and/or a medication, etc.) and/or one or more lumens 206a, 206b, . . . 206n (e.g., fluid lines for delivering a fluid and/or a medication to a patient, etc.) of one or more catheters. For example, the one or more sensors 202a, 202b, . . . 202n may be configured to sense, detect, and/or determine pathway data associated with the one or more medication delivery pathways 203a, 203b, . . . 203n (e.g., pathway data associated with the one or more ports 204a, 204b, . . . 204n and/or the one or more lumens 206a, 206b, . . . 206n of the one or more catheters, etc.) and/or one or more medications delivered or being delivered to a patient via the one or more medication delivery pathways 203a, 203b, . . . 203n (e.g., via the one or more ports 204a, 204b, . . . 204n and/or the one or more lumens 206a, 206b, . . . 206n of the one or more catheters, etc.). However, non-limiting embodiments or aspects are not limited thereto, and medication delivery pathway system 104 may include any number of medication delivery pathways, such as a plurality of medication delivery pathways included in an IV set formed from a confluence of multiple connectors and lumens or lines and ending in a single catheter. For example, different drugs can be injected into different pathway branches of the IV set. As an example, and as described in more detail herein below with respect to FIGS. 4 and 5, medication administration system 102 may determine, according to a rule set, based on pathway data and prescription data, a least incompatible path (e.g., a most compatible path, etc.) to the catheter, a shortest path to the catheter (or longest path to the catheter if the medication needs to be diluted by a main fluid running through the IV set), a set of branches that were flushed, and/or a set of branches with the most compatible fluids and/or flow rates with respect to a medication.

In some non-limiting embodiments or aspects, medication delivery pathway sensor system 104 may include one or more of the systems and/or devices described by international application no. PCT/US2020/016539, filed on Feb. 4, 2020, and assigned to the assignee of the present application, the entire contents of which are hereby incorporated by reference. For example, the one or more sensors 202a, 202b, 202n may include one or more smart devices as described by international application no. PCT/US2020/016539.

In some non-limiting embodiments or aspects, medication administration system 102, medication delivery pathway sensor system 104, and/or remote computing system 108 may include one or more devices capable of receiving, storing, processing, and/or providing pathway data associated with a medication delivery pathway for delivering medication to a patient. For example, pathway data may define or identify a medication delivery pathway for delivering medication to a patient (e.g., each medication delivery pathway for delivering medication to the patient, etc.) and/or one or more attributes of (e.g., metadata associated with) the medication delivery pathway. As an example, an attribute of a medication delivery pathway may include at least one of the following: a patient to which the medication delivery pathway is connected, a location of a body of a patient to which the medication delivery pathway is configured to deliver medication, a catheter of the medication delivery pathway, a lumen of the medication delivery pathway, a port of the medication delivery pathway, a type of the medication delivery pathway (e.g., a peripheral intravenous line (PIV), a peripherally inserted central catheter (PICC), a central catheter, a midline catheter, etc.), a material of the medication delivery pathway, a medication previously delivered or currently being delivered via the medication delivery pathway, a time at which a medication was previously delivered or is currently being delivered via the medication delivery pathway, a flushing event performed in the medication delivery pathway, a time at which a flushing event occurred in the medication delivery pathway, a distance (e.g., lumen or line length, etc.) of a port of a medication delivery pathway from a patient, a light property of a medication delivery pathway (e.g., whether a tubing is amber and/or otherwise opaque to protect light sensitive drugs, whether a tubing a translucent, etc.), or any combination thereof. In some non-limiting embodiments or aspects, pathway data may include prescription data. For example, pathway data identify each medication delivery pathway (e.g., each catheter, lumen, port, etc.) associated with a patient and include a history of each medication that was delivered or is being delivered through each medication delivery pathway (e.g., what medications were delivered through what catheters, lumens, and/or ports, etc.) to the patient (e.g., pumped medications, piggybacked medications, drip medications, blood draws, etc.) and/or each medication that was delivered or is being delivered to the patient via another delivery route (e.g., injection, oral, etc.).

In some non-limiting embodiments or aspects, medication administration system 102, medication delivery pathway sensor system 104, and/or remote computing system 108 may include one or more devices capable of receiving, storing, processing, and/or providing prescription data associated with a medication delivered, being delivered, or prescribed to be delivered to a patient. For example, prescription data may define or identify a medication delivered, being delivered, or prescribed to be delivered to a patient (e.g., each medication delivered (e.g., a prior medication administration), being delivered (e.g., a current or ongoing medication administration), or prescribed to be delivered to the patient (e.g., a new or future medication administration), etc.) and/or one or more attributes of (e.g., metadata associated with) the medication. As an example, an attribute of a medication may include at least one of the following: a delivery type of the medication (e.g., a push delivery medication, a drip delivery medication, a pump delivery medication, etc.), a dosage amount of the medication, a dosage rate of the medication, a time period acceptable for delivering the medication to a patient, or any combination thereof.

In some non-limiting embodiments or aspects, medication administration system 102, medication delivery pathway sensor system 104, and/or remote computing system 108 may include one or more devices capable of receiving, storing, processing, and/or providing rule set data associated with at least one rule set including at least one rule, which when applied to pathway data and/or prescription data associated with a patient, dictates or determines one or more medication delivery pathways to be used for delivering one or more medications to the patient. For example, a rule may define, dictate, or determine at least one of the following: whether (e.g., a time after which, etc.) a medication is compatible to be delivered to a patient via a same medication delivery pathway as another medication, whether (e.g., a time after which, etc.) a medication including an attribute (e.g., a chemical property, a physical property, an osmolality of drug, a pH, etc.) is compatible to be delivered to a patient via a same medication delivery pathway as another medication including another attribute (e.g., another chemical property, another physical property, another osmolality of drug, another pH, etc.), whether (e.g., a volume of flushing after which, etc.) a medication is compatible to be delivered to a patient via a same medication delivery pathway as another medication if the same medication delivery pathway is flushed between deliveries of the medication and the another medication, whether a medication is compatible to be delivered to a patient via a type of a medication delivery pathway, whether a medication is compatible to be delivered to a patient at a location of a body of the patient, whether a medication is compatible to be delivered to a patient via a medication delivery pathway including a material, an amount of time a medication is compatible to be delivered to a patient via a medication delivery pathway including a material, whether (e.g., a time between which, etc.) a medication is compatible to be delivered to the patient with another medication, a priority of delivery of a medication with respect to another medication (e.g., “stat” drugs may have a higher priority for delivery than drugs associated with a defined acceptable time window, etc.), a priority of delivery of a type of medication (e.g., a Red vesicant, a Yellow vesicant, or a G vesicant, a critical medication vs. a non-critical medication, etc.) via a type, material, and/or a location of medication delivery pathway (e.g., a catheter, lumen, and/or port type, color, size, and/or patient location, etc.) with respect to another type, material, and/or location of medication delivery pathway (e.g., hospital preferences, patient preferences, manufacturer preferences and/or the like may dictate that a particular type of medication delivery pathway be used over or before another type of medication delivery pathway, etc.), whether to prompt a user to flush a medication delivery pathway before and/or after delivering a medication via the medication delivery pathway, whether to prompt a user to install a new medication delivery pathway for delivering a medication to a patient, whether to determine an existing medication delivery pathway for delivering a medication to a patient, whether a concentration of a medication is compatible to be delivered to a patient via a same medication delivery pathway as a concentration of another medication, whether a diluent associated with a medication is compatible to be delivered to a patient via a same medication delivery pathway as diluent associated with another medication, whether a delivery vehicle of a medication is compatible to be delivered to a patient via a same medication delivery pathway as a delivery vehicle of another medication, or any combination thereof.

In some non-limiting embodiments or aspects, vesicants may be divided into groups or lists based on amounts of irritation likely to be caused by the vesicants, potentials of the vesicants to cause site reactions, irritation, and/or tissue or vein damage (e.g., phlebitis, etc.). For example, the groups or lists of vesicants may be hospital specific and/or more generally detailed and standardized. As an example, vesicants may be divided into the following groups: red (R) vesicants, green (G) vesicants, and yellow (Y) vesicants. In such an example, R vesicants may be most likely to cause irritation and/or site reactions, G vesicants may be least likely to cause irritation and/or site reactions, and Y vesicants may have a likelihood of causing irritation and/or site reactions that is somewhere between the R vesicants and the G vesicants.

Communication network 106 may include one or more wired and/or wireless networks. For example, communication network 106 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or any combination of these or other types of networks.

Remote computing system 108 may include one or more devices capable of receiving information and/or data from medication administration system 102 and/or medication delivery pathway sensor system 104 (e.g., via communication network 112, etc.) and/or communicating information and/or data to medication administration system 102 and/or medication delivery pathway sensor system 104 (e.g., via communication network 112, etc.). For example, remote computing system 108 may include one or more computing systems including one or more processors (e.g., one or more computing devices, one or more mobile computing devices, one or more servers, etc.). In some non-limiting embodiments or aspects, remote computing system 114 includes a nurse station in a hospital. In some non-limiting embodiments or aspects, remote computing system 108 includes a secure hospital server and/or one or more secure hospital databases that store personally identifiable information (PII) and/or Health Insurance Portability and Accountability Act (HIPAA) protected information.

The number and arrangement of systems and devices shown in FIGS. 1 and 2 are provided as an example. There can be additional systems and/or devices, fewer systems and/or devices, different systems and/or devices, or differently arranged systems and/or devices than those shown in FIGS. 1 and 2. Furthermore, two or more systems or devices shown in FIGS. 1 and 2 can be implemented within a single system or a single device, or a single system or a single device shown in FIGS. 1 and 2 can be implemented as multiple, distributed systems or devices. Additionally, or alternatively, a set of systems or a set of devices (e.g., one or more systems, one or more devices, etc.) of environment 100 can perform one or more functions described as being performed by another set of systems or another set of devices of environment 100.

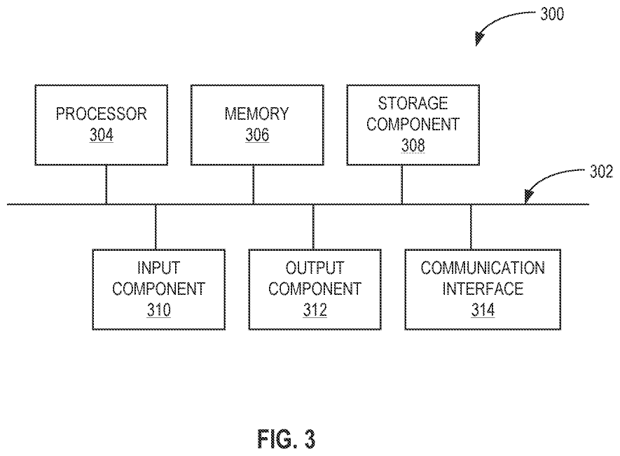

Referring now to FIG. 3, FIG. 3 is a diagram of example components of a device 300. Device 300 may correspond to one or more devices of reader device 102 and/or one or more devices of remote computing system 114. In some non-limiting embodiments or aspects, one or more devices of reader device 102 and/or one or more devices of remote computing system 114 can include at least one device 300 and/or at least one component of device 300. As shown in FIG. 3, device 300 may include a bus 302, a processor 304, memory 306, a storage component 308, an input component 310, an output component 312, and a communication interface 314.

Bus 302 may include a component that permits communication among the components of device 300. In some non-limiting embodiments or aspects, processor 304 may be implemented in hardware, firmware, or a combination of hardware and software. For example, processor 304 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), a microcontroller (MCU), etc.) that can be programmed to perform a function. Memory 306 may include random access memory (RAM), read only memory (ROM), and/or another type of dynamic or static storage device (e.g., flash memory, magnetic memory, optical memory, etc.) that stores information and/or instructions for use by processor 304.

Storage component 308 may store information and/or software related to the operation and use of device 300. For example, storage component 308 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 310 may include a component that permits device 300 to receive information, such as via user input (e.g., a touch screen display, a keyboard, a keypad, a mouse, a button, a switch, a microphone, a camera, an electroencephalogram (EEG) monitor, patient monitoring system etc.). Additionally, or alternatively, input component 310 may include a sensor for sensing information (e.g., a global positioning system (GPS) component, an accelerometer, a gyroscope, an actuator, etc.). Output component 312 may include a component that provides output information from device 300 (e.g., a display, a speaker, one or more light-emitting diodes (LEDs), and/or the like).

Communication interface 314 may include a transceiver-like component (e.g., a transceiver, a separate receiver and transmitter, etc.) that enables device 300 to communicate with other devices, such as via a wired connection, a wireless connection, or a combination of wired and wireless connections. Communication interface 314 may permit device 300 to receive information from another device and/or provide information to another device. For example, communication interface 314 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 300 may perform one or more processes described herein. Device 300 may perform these processes based on processor 304 executing software instructions stored by a computer-readable medium, such as memory 306 and/or storage component 308. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 306 and/or storage component 308 from another computer-readable medium or from another device via communication interface 314. When executed, software instructions stored in memory 306 and/or storage component 308 may cause processor 304 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 306 and/or storage component 308 may include data storage or one or more data structures (e.g., a database, etc.). Device 300 may be capable of receiving information from, storing information in, communicating information to, or searching information stored in the data storage or one or more data structures in memory 306 and/or storage component 308. For example, the information may include pathway data, prescription data, rule set data, or any combination thereof.

The number and arrangement of components shown in FIG. 3 are provided as an example. In some non-limiting embodiments or aspects, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

Referring now to FIG. 4, FIG. 4 is a flowchart of a non-limiting embodiment or aspect of a process 400 for medication administration. In some non-limiting embodiments or aspects, one or more of the steps of process 400 are performed (e.g., completely, partially, etc.) by medication administration system 102 (e.g., one or more devices of a system of medication administration system 102, etc.). In some non-limiting embodiments or aspects, one or more of the steps of process 400 are performed (e.g., completely, partially, etc.) by another device or a group of devices separate from or including medication administration system 102, such as medication delivery pathway sensor system 104 (e.g., one or more devices of medication delivery pathway sensor system 104, etc.) and/or remote computing system 108 (e.g., one or more devices of remote computing system 108, etc.).

As shown in FIG. 4, at step 402, process 400 includes obtaining pathway data associated with a delivery pathway for delivering medication to a patient. For example, medication administration system 102 may obtain pathway data associated with a medication delivery pathway for delivering medication to a patient. As an example, medication administration system 102 may obtain pathway data associated with at least one medication delivery pathway 203a, 203b, . . . 203n for delivering medication to a patient (e.g., pathway data associated with each medication delivery pathway for delivering medication to the patient, etc.).

In some non-limiting embodiments or aspects, medication administration system 102 may obtain pathway data from at least one sensor coupled to at least one medication delivery pathway. For example, medication administration system 102 may obtain pathway data from medication delivery pathway sensor system 104. As an example, medication administration system 102 may obtain pathway data from the one or more sensors 202a, 202b, . . . 202n coupled to the one or more medication delivery pathways 203a, 203b, . . . 203n. In such an example, the at least one other medication may be delivered or may be being delivered via the at least one medication delivery pathway, and the one or more medications associated may be prescribed for delivery to the patient. However, non-limiting embodiments or aspects are not limited thereto, and medication administration system 102 may obtain pathway data input by one or more users to one or more user interfaces of medication administration system 102, medication delivery pathway sensor system 104, and/or remote computing system 108.

In some non-limiting embodiments or aspects, medication administration system 102 may communicate (e.g., via user interface, etc.) the pathway data associated with a patient to a pharmacist at a time of a medication order, which may enable the pharmacist to have firsthand knowledge of what medications have been through which medication delivery pathways for a patient, as well as proactively provide guidance to a health practitioner via a label associated with the medication order.

As shown in FIG. 4, at step 404, process 400 includes obtaining prescription data associated with a medication prescribed for delivery to a patient. For example, medication administration system 102 may obtain prescription data associated with a medication prescribed for delivery to a patient (e.g., prescription data associated with each medication prescribed for delivery to the patient, etc.). As an example, medication administration system 102 may obtain prescription data associated with (i) one or more medications prescribed for delivery to the patient and (ii) at least one other medication delivered, being delivered, or prescribed to be delivered to the patient.

As shown in FIG. 4, at step 406, process 400 includes determining, according to a rule set, based on pathway data and/or prescription data, a medication delivery pathway for delivery of a medication to a patient. For example, medication administration system 102 may determine, according to a rule set, based on pathway data and prescription data, a medication delivery pathway for delivery of a medication to a patient. As an example, medication administration system 102 may determine, according to at least one rule set, based on the pathway data and the prescription data, one or more medication delivery pathways 203a, 203b, . . . 203n for delivery of the one or more medications to the patient. In such an example medication administration system 102 may determine the one or more medication delivery pathways for delivery of the one or more medication to the patient by applying at least one rule of the at least one rule set to the pathway data and/or the prescription data.

In some non-limiting embodiments or aspects, a user may selectively determine, for use in determining a medication delivery pathway for delivery of a medication to a patient, one or more rule sets and/or one or more rules of the one or more rule sets from a plurality of available rule sets including a plurality of available rules. For example, a hospital may have an option to determine which medication compatibility rule sets and/or rules are used to drive decisions on delivery pathway compatibility.

Further details regarding non-limiting embodiments or aspects of step 406 of process 400 are provided below with regard to FIG. 5.

As shown in FIG. 4, at step 408, process 400 includes assigning a medication for delivery to a patient via a medication delivery pathway. For example, medication administration system 102 may assign (e.g., assign, recommend, alert, etc.) a medication for delivery to a patient via a medication delivery pathway. As an example, medication administration system 102 may assign the one or more medications for delivery to the patient via the one or more medication delivery pathways 203a, 203b, . . . 203n (e.g., via an update or assignment in medication administration system 102, e.g., via an assignment or recommendation to a user, etc.). In such an example, after assigning the one or more medications for delivery to the patient via the one or more medication delivery pathways, processing may return to step 402, in which medication administration system 102 may obtain updated pathway data associated with delivered and/or currently being delivered medications, and to step 404, in which medication administration system 102 may obtain further prescription data associated with one or more further medications prescribed for delivery to the patient. It is contemplated herein that multiple acceptable ports may be identified for a single medication.

In some non-limiting embodiments or aspects, medication administration system 102 may assign the one or more medications for delivery to the patient via an oral medication delivery pathway. For example, medication administration system 102 may obtain pathway data associated with oral administration of a medication to a patient, obtain prescription data associated with one or medications prescribed for delivery to a patient, determine, according to a rule set, based on the pathway data and the prescription data, an oral medication delivery pathway for delivery of the one or medications to the patient, and assign the one or more medications for delivery to the patient via the oral administration pathway.

In some non-limiting embodiments or aspects, medication administration system 102 may communicate to one or more users (e.g., to a pharmacist, a health practitioner, etc.) the assignment of the one or more medications for delivery to the patient via the one or more medication delivery pathways and/or one or more time periods for delivery of the one or more medications to the patient via the one or medication delivery pathways. In some non-limiting embodiments or aspects, medication administration system 102 may communicate to the one or more users post medication administration guidance, such as one or more physiological parameters of the patient to monitor post medication administration, one or more post administration procedures to perform (e.g., medication wasting, disposal, logging, etc.), and/or the like, based on a type of the one or more medications assigned for delivery to the patient via the one or more medication delivery pathways.

In some non-limiting embodiments or aspects, medication administration system 102 may prompt a user to flush a medication delivery pathway when assigning a medication for delivery to a patient via the medication delivery pathway. For example, medication administration system 102 may prompt a user to flush the one or more medication delivery pathways at least one of before and after delivering the one or more medications via the one or more medication delivery pathways (e.g., in response to a determination that whether the one or more medications are compatible to be delivered to the patient via the same medication delivery pathway as the at least one medication if the same medication delivery pathway is flushed in between deliveries of the one or more medications and the at least one medication as described in more detail below with respect to FIG. 5, etc.). In such an example, the one or more medication delivery pathways include the at least one medication delivery pathway for delivering medication to the patient.

In some non-limiting embodiments or aspects, medication administration system 102 may prompt a user to install a medication delivery pathway when assigning a medication for delivery to a patient via the medication delivery pathway. For example, medication administration system 102 may prompt prompting a user to install the one or more medication delivery pathways at a body of the patient (e.g., in response to one or more determinations that the one or more medications are not compatible with the at least one medication delivery pathway for delivering medication to the patient as described in more detail below with respect to FIG. 5, etc.).

Referring now to FIG. 5, FIG. 5 is a flowchart of a non-limiting embodiment or aspect of a process 500 for medication administration. In some non-limiting embodiments or aspects, one or more of the steps of process 500 are performed (e.g., completely, partially, etc.) by medication administration system 102 (e.g., one or more devices of a system of medication administration system 102, etc.). In some non-limiting embodiments or aspects, one or more of the steps of process 500 are performed (e.g., completely, partially, etc.) by another device or a group of devices separate from or including medication administration system 102, such as medication delivery pathway sensor system 104 (e.g., one or more devices of medication delivery pathway sensor system 104, etc.) and/or remote computing system 108 (e.g., one or more devices of remote computing system 108, etc.).

As shown in FIG. 5, at step 502, process 500 includes determining, according to a rule set, based on pathway data and/or prescription data, whether medications are compatible to be delivered to the patient via a same medication delivery pathway. For example, medication administration system 102 may determine, according to a rule set, based on pathway data and/or prescription data, whether medications are compatible to be delivered to the patient via a same medication delivery pathway. As an example, medication administration system 102 may determine, according to the at least one rule set, based on the pathway data and the prescription data, whether the one or more medications are compatible to be delivered to the patient via a same medication delivery pathway as the at least one medication based on the pathway data and the prescription data.

As shown in FIG. 5, at step 504, process 500 includes determining, according to a rule set, based on pathway data and/or prescription data, whether medications are compatible to be delivered to a patient via a same medication delivery pathway if the same medication delivery pathway is flushed in between deliveries of the medications. For example, medication administration system 102 may determine, according to a rule set, based on pathway data and/or prescription data, whether medications are compatible to be delivered to a patient via a same medication delivery pathway if the same medication delivery pathway is flushed in between deliveries of the medications. As an example, medication administration system 102 may determine, according to the at least one rule set, based on the pathway data and the prescription data, whether the one or more medications are compatible to be delivered to the patient via the same medication delivery pathway as the at least one medication if the same medication delivery pathway is flushed in between deliveries of the one or more medications and the at least one medication.

As shown in FIG. 5, at step 506, process 500 includes segmenting, according to the at least one rule set, each medication prescribed for delivery to the patient into a plurality of medication groups based on pathway data and/or the prescription data. For example, medication administration system 102 may segment, according to the at least one rule set, each medication prescribed for delivery to the patient into a plurality of medication groups based on pathway data and/or the prescription data. As an example, medication administration system 102 may segment, according to the at least one rule set, the one or more medications and the at least one medication into a plurality of medication groups based on whether the one or more medications and the at least one medication are compatible to be delivered to the patient via the same medication delivery pathway and/or whether the one or more medications are compatible to be delivered to the patient via the same medication delivery pathway as the at least one medication if the same medication delivery pathway is flushed in between deliveries of the one or more medications and the at least one medication. In such an example, medication administration system 102 may segment medications in a same prescription or order based on compatibilities of the medications with each other and/or to create the largest groups of compatible medications for existing medication delivery pathways for the patient.

As shown in FIG. 5, at step 508, process 500 includes determining, according to a rule set, based on pathway data and/or prescription data, whether a medication is compatible to be delivered to a patient via a type of a medication delivery pathway. For example, medication administration system 102 may determine, based on pathway data and/or prescription data, whether a medication is compatible to be delivered to a patient via a type of a medication delivery pathway. As an example, medication administration system 102 may determine, according to the at least one rule set, based on the pathway data and the prescription data, whether the one or more medications are compatible to be delivered to the patient via the one or more types of the at least one medication delivery pathway.

As shown in FIG. 5, at step 510, process 500 includes determining, according to a rule set, based on pathway data and/or prescription data, whether a medication is compatible to be delivered to a patient at a location of a body of the patient to which a medication delivery pathway is configured to deliver medication. For example, medication administration system 102 may determine, according to a rule set, based on pathway data and/or prescription data, whether a medication is compatible to be delivered to a patient at a location of a body of the patient to which a medication delivery pathway is configured to deliver medication. As an example, medication administration system 102 may determine, according to the at least one rule set, based on the pathway data and the prescription data, whether the one or more medications are compatible to be delivered to the patient at the one or more locations of the body of the patient.

As shown in FIG. 5, at step 512, process 500 includes determining, according to a rule set, based on pathway data and/or prescription data, whether a medication is compatible to be delivered to a patient via a medication delivery pathway including a material. For example, medication administration system 102 may determine, according to a rule set, based on pathway data and/or prescription data, whether a medication is compatible to be delivered to a patient via a medication delivery pathway including a material. As an example, medication administration system 102 may determine, according to the at least one rule set, based on the pathway data and the prescription data, whether the one or more medications are compatible to be delivered to the patient via a pathway including the one or more materials.

As shown in FIG. 5, at step 514, process 500 includes determining, according to a rule set, based on pathway data and/or prescription data, an amount of time a medication is compatible to be delivered to a patient via a pathway including one or more materials. For example, medication administration system 102 may determine, according to a rule set, based on pathway data and/or prescription data, an amount of time a medication is compatible to be delivered to a patient via a pathway including one or more materials. As an example, medication administration system 102 may determine, according to the at least one rule set, based on the pathway data and the prescription data, an amount of time the one or more medications are compatible to be delivered to the patient via the pathway including the one or more materials.

As shown in FIG. 5, at step 516, process 500 includes determining, according to a rule set, based on pathway data and/or prescription data, a time period for delivery of a medication to a patient via a medication delivery pathway. For example, medication administration system 102 may determine, according to a rule set, based on pathway data and/or prescription data, a time period for delivery of a medication to a patient via a medication delivery pathway. As an example, medication administration system 102 may determine, according to the at least one rule set (e.g., according to a priority of delivery of a medication with respect to another medication, a priority of delivery of a type of medication via a type, material, and/or location of a medication delivery pathway, etc.), based on the pathway data and the prescription data, one or more first time periods for delivery of the one or more medications via the one or more medication delivery pathways. In such an example, prescription data may be associated with one or more delivery types of the one or more medications, one or more dosage amounts of the one or more medications, one or more dosage rates of the one or more medications, one or more time periods acceptable for delivering the one or more medications to the patient, or any combination thereof. In some non-limiting embodiments or aspects, medication administration system 102 may assign medications to available medication delivery pathways according to the following order of priority: 1) medications associated with a shortest available time window for delivery, 2) vesicants R, then Y, then G, 3) according to a medication group size, and 4) alphabetically according to medication name. Accordingly, medication administration system 102 may determine and provide a timeline of delivery for the one or more medications to the patient and the medication delivery pathways through which the one or more medications are assigned to be delivered.

As shown in FIG. 5, at step 518, process 500 includes determining, according to a rule set, based on pathway data and/or prescription data, a second time period after a first time period for delivery of a second dose of a medication to a patient via a medication delivery pathway. For example, medication administration system 102 may determine, according to a rule set, based on pathway data and/or prescription data, a second time period after a first time period for delivery of a second dose of a medication to a patient via a medication delivery pathway. As an example, medication administration system 102 may determine, according to the at least one rule set, based on the pathway data and the prescription data, one or more second time periods after the one or more first time periods for delivery of one or more second doses of the one or more medications via the one or more medication delivery pathways. Accordingly, medication administration system 102 may determine re-timing of the one or more medications and/or provide the determined re-timing (e.g., directly to a pharmacist upon order of the one or more medications, etc.) to reduce a need for placement of additional medication delivery pathways (e.g., additional catheters, etc.) and/or to reduce medication incompatibilities resulting in medication delivery pathway issues, such as precipitate formation in a catheter line, and/or the like.

As shown in FIG. 5, at step 520, process 500 includes determining, based on one or more of the determinations in steps 502-518, a medication delivery pathway (e.g., a catheter, a lumen, a port, etc.) for delivery of the medication to the patient. For example, medication administration system 102 may determine, according to a rule set, based on one or more of the determinations in steps 502-518, a medication delivery pathway for delivery of the medication to the patient. As an example, medication administration system 102 may determine, according to at least one rule in the at least one rule set applied to the one or more of the determinations in steps 502-518, one or more medication delivery pathways for delivery of the one or more medications to the patient. For example, medication administration system may determine, according to one or more of the determinations in steps 502-518, one or more medication delivery pathways for delivery of the plurality of medication groups to the patient based on the pathway data and the prescription data. As an example, medication delivery system 102 may determine, according to a rule set, based on one or more of the determinations in steps 502-518, a medication delivery pathway for delivery of the medication to the patient from a group of existing medication delivery pathways (e.g., already installed medication delivery pathways, etc.) and/or from a group of available medication delivery pathways (e.g., medication delivery pathways available to be installed by a user, etc.) that provides a least incompatible medication delivery pathway (e.g., a most compatible medication delivery pathway, etc.), a shortest length medication delivery pathway to the patient (or a longest length medication delivery pathway to the patient if the medication needs to be diluted by a primary and/or secondary infusion fluids), a set of branches or paths that were flushed, a set of branches with the most compatible fluids and/or flow rates, or any combination thereof.

In some non-limiting embodiments or aspects, medication administration system 102 may provide assignments of the one or more medications for delivery via the one or more medication delivery pathways via a user interface. For example, the user interface may include a visual map of the medication delivery pathways for the patient and instructions indicating which of the medication delivery pathways to use for delivering which medications and/or time periods at which the mediations are to be delivered, and/or instructions indicating where to insert or install any new medication delivery pathways determined for delivery of the one or more medications to the patient.

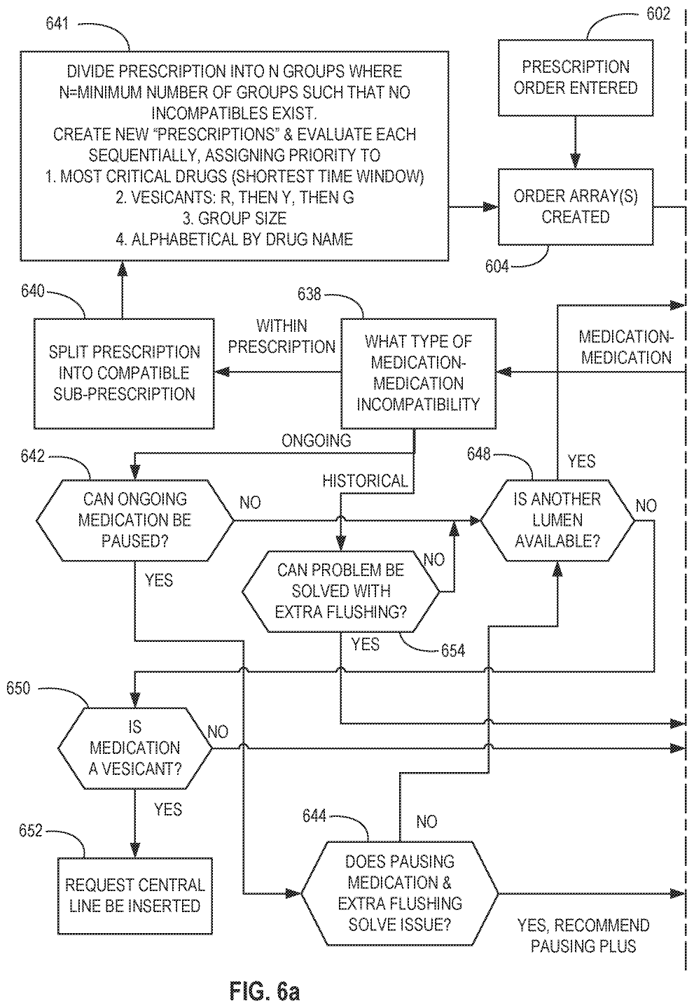

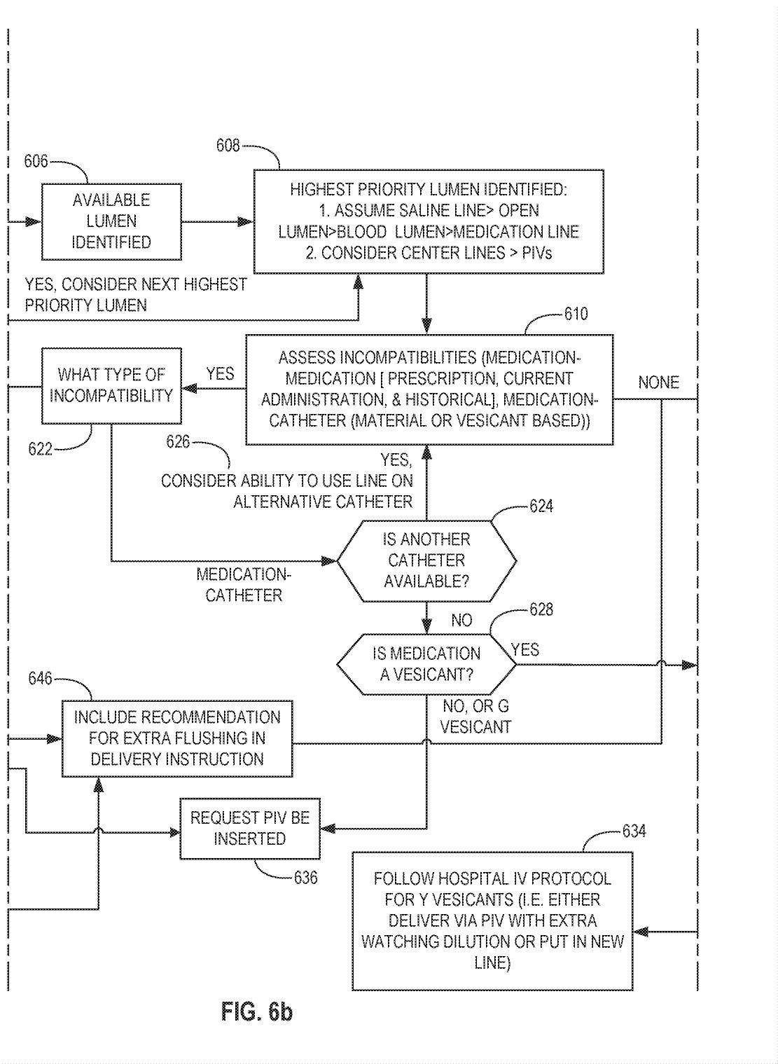

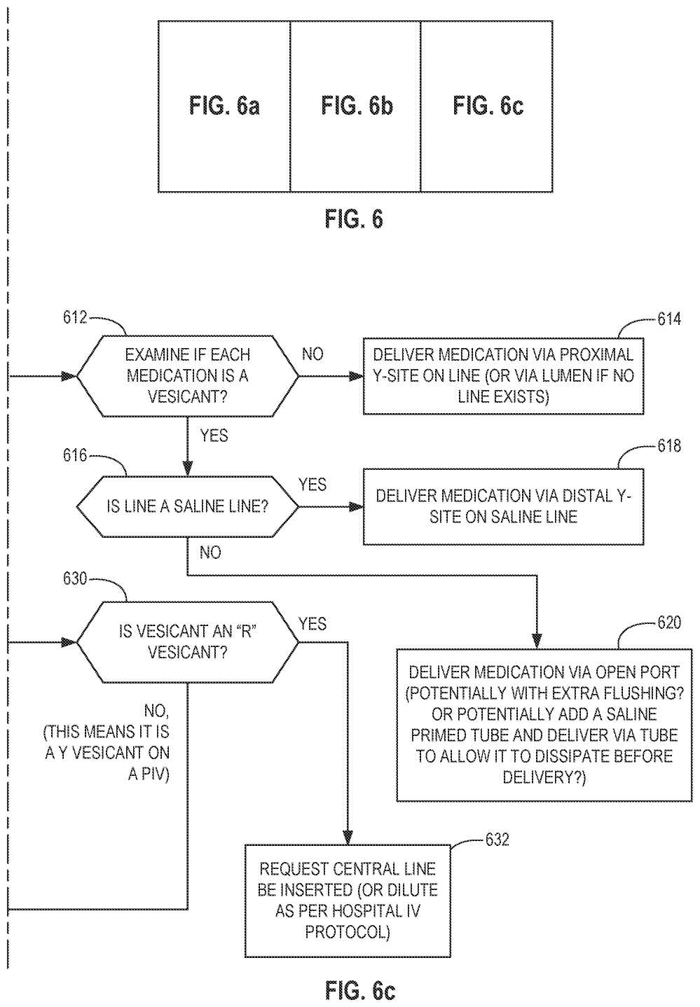

Referring now to FIGS. 6a-6c, FIGS. 6a-6c are a flowchart of an overview of a non-limiting embodiment or aspect of an implementation 600 relating to a process for medication administration.

As shown by reference number 602 in FIGS. 6a-6c, medication administration system 102 may receive prescription data associated with medications prescribed for a patient in a prescription. As shown by reference number 604 in FIGS. 6a-6c, medication administration system 102 may create order arrays based on the prescription data. As shown by reference number 606 in FIGS. 6a-6c, medication administration system 102 may identify available medication delivery pathways for the patient. As shown by reference number 608 in FIGS. 6a-6c, medication administration system 102 may identify a highest priority medication delivery pathway. As shown by reference number 610 in FIGS. 6a-6c, medication administration system 102 may determine if there are any incompatibilities between medications in the prescription and/or if there are any incompatibilities between medications in the prescription and the medication delivery pathways for the patient.

If, at reference number 610, medication administration system 102 determines that there are not any incompatibilities, at reference number 612, medication administration system 102 may determine if each medication is a vesicant. If, at reference number 612, medication administration system 102 determines that none of the medications are a vesicant, at reference number 614, medication administration system 102 may assign the medications for delivery via existing medication delivery pathways (e.g., via a proximal y-site on line (or via a lumen if no line exists), etc.). If, at reference number 612, medication administration system 102 determines that the medications include a vesicant, at reference number 616, medication administration system 102 may determine if the existing medication administration pathways include a saline line. If, at reference number 616, medication administration system 102 determines that the existing medication administration pathways include a saline line, at reference number 618, medication administration system 102 may assign the vesicant for delivery via a distal y-site on the saline line. If, at reference number 616, medication administration system 102 determines that the existing medication administration pathways do not include a saline line, at reference number 620, medication administration system 102 may assign the vesicant for delivery via an open port.

If, at reference number 610, medication administration system 102 determines that there are incompatibilities, at reference number 622, medication administration system 102 may determine a type of the incompatibility. If, at reference number 622, medication administration system 102 determines that the type of the incompatibility is an incompatibility between a medication and a medication delivery pathway, at reference number 624, medication administration system 102 may determine if another compatible medication delivery pathway is available. If, at reference number 624, medication administration system 102 determines that another compatible medication delivery pathway is available, at reference number 626, medication administration system 102 may assign the medication for delivery via the another medication delivery pathway. If, at reference number 624, mediation administration system 102 determines that another compatible medication delivery pathway is not available, at reference number 628, medication administration system 102 may determine if the medication includes a vesicant.

If, at reference number 628, medication administration system 102 determines that the medication is a vesicant (and/or not a G vesicant), at reference number 630, medication administration system 102 determines if the vesicant is an R vesicant. If, at reference number 630, medication administration system 102 determines that the medication is an R vesicant, at reference number 632, medication administration system 102 prompts the user to insert or install a central line and assigns the R vesicant for delivery via the central line. If, at reference number 630, medication administration system 102 determines that the medication is not an R vesicant (e.g., that the medication is a Y vesicant), at reference number 634, medication administration system 102 may prompt the user to follow hospital protocol for delivering a Y vesicant and assign the Y vesicant for delivery via a PIV with extra flushing or via a newly installed medication delivery pathway. If, at reference number 628, medication administration system 102 determines that the medication is not a vesicant (or a G vesicant), at reference number 636, medication administration system 102 may prompt the user to install a new medication delivery pathway (e.g., a PIV, etc.) and assign the medication for delivery via the newly installed medication delivery pathway.

If, at reference number 622, medication administration system 102 determines that the type of the incompatibility is an incompatibility between a medication and another medication, at reference number 638, medication administration system 102 may determine a type of the medication-medication incompatibility. If, at reference number 638, medication administration system 102 determines that the type of incompatibility is an incompatibility between medications in the same prescription, at reference number 640, medication administration system 102 may segment the medications in the prescription into groups or sub-prescriptions of compatible medications. For example, at reference number 642, medication delivery system 102 may divide the prescription into N groups where N is a minimum number of groups such that no incompatibles between medications in those groups exist. As an example, medication administration system 102 may create new prescriptions and evaluate each new prescription sequentially. In such an example, administration system 102 may assign medications to available medication delivery pathways according to the following order of priority: 1) medications associated with a shortest available time window for delivery, 2) vesicants R, then Y, then G, 3) according to a medication group size, and 4) alphabetically according to medication name.

If, at reference number 638, medication administration system 102 determines that the type of incompatibility is an incompatibility between a medication in prescription and an ongoing medication (e.g., a medication currently being delivered to the patient, etc.), at reference number 642, medication administration system 102 may determine if delivery of the ongoing medication can be paused. If, at reference number 642, medication administration system 102 determines that the ongoing medication can be paused, at reference number 644, medication administration system 102 may determine if pausing the ongoing medication and performing flushing of the medication delivery pathway renders the medication delivery pathway compatible for the medication in the prescription. If, at reference number 644, medication administration system 102 determines that pausing the ongoing medication and performing the extra flushing achieves compatibility, at reference number 646, medication administration system 102 may include an instructions to perform flushing of the medication delivery pathway to which the medication is assigned and processing may return to reference number 612 to determine the medication delivery pathway to assign to the medication.

If, at reference number 642, medication administration system 102 determines that the ongoing medication cannot be paused, at reference number 648, medication administration system 102 may determine if another medication delivery pathway (e.g., another lumen, etc.) is available for medication delivery to the patient. If, at reference number 648, medication administration system 102 determines that another medication delivery pathway is available for medication delivery to the patient, processing may return to reference number 608 for medication administration system 102 to consider a next highest priority medication delivery pathway for delivery of the medication to the patient. If, at reference number 648, medication administration system 102 determines that another medication delivery pathway is not currently available for medication delivery to the patient, at reference number 650, medication administration system 102 may determine if the medication includes a vesicant. If, at reference number 650, medication administration system 102 determines that the medication does not include a vesicant, processing may return to reference number 636 at which medication administration system 102 may prompt the user to install a new medication delivery pathway (e.g., a PIV, etc.) and assign the medication for delivery via the newly installed medication delivery pathway. If, at reference number 650, medication administration system 102 determines that the medication includes a vesicant, at reference number 652, medication administration system 102 may prompt the user to inserts or install a new medication delivery pathway (e.g., a central line, etc.) for delivery of the vesicant.

If, at reference number 638, medication administration system 102 determines that the type of incompatibility is an incompatibility between a medications in the prescription and a medication previously delivered via the same medication delivery pathway, at reference number 654, medication administration system 102 may determine if performing flushing of the medication delivery pathway renders the medication delivery pathway compatible for the medication in the prescription. If, at reference number 654 medication administration system 102 determines that flushing of the medication delivery pathway renders the medication delivery pathway compatible for the medication in the prescription, processing may return to reference number 646 at which medication administration system 102 may include an instructions to perform flushing of the medication delivery pathway to which the medication is assigned and processing may return to reference number 612 to determine the medication delivery pathway to assign to the medication. If, at reference number 654 medication administration system 102 determines that flushing of the medication delivery pathway does not render the medication delivery pathway compatible for the medication in the prescription, processing may return to reference number 648 at which medication administration system 102 may determine if another medication delivery pathway (e.g., another lumen, etc.) is available for medication delivery to the patient.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A computer-implemented method, comprising:
obtaining, with at least one processor, pathway data associated with at least one medication delivery pathway for delivering medication to a patient, wherein the at least one medication delivery pathway includes at least one of the following: at least one catheter, at least one lumen, at least one port, or any combination thereof;
obtaining, with at least one processor, prescription data associated with (i) one or more medications prescribed for delivery to the patient and (ii) at least one other medication delivered, being delivered, or prescribed to be delivered to the patient;
determining, with at least one processor, according to at least one rule set, based on the pathway data and the prescription data, one or more medication delivery pathways for delivery of the one or more medications to the patient; and
assigning, with at least one processor, the one or more medications for delivery to the patient via the one or more medication delivery pathways.

2. The computer-implemented method of claim 1, wherein determining the one or more medication delivery pathways includes:
determining, according to the at least one rule set, based on the pathway data and the prescription data, whether the one or more medications are compatible to be delivered to the patient via a same medication delivery pathway as the at least one medication based on the pathway data and the prescription data.

3. The computer-implemented method of claim 2, wherein determining whether the one or more medications are compatible to be delivered to the patient via the same medication delivery pathway as the at least one medication includes:
determining, according to the at least one rule set, based on the pathway data and the prescription data, whether the one or more medications are compatible to be delivered to the patient via the same medication delivery pathway as the at least one medication if the same medication delivery pathway is flushed in between deliveries of the one or more medications and the at least one medication.

4. The computer-implemented method of claim 3, wherein the one or more medication delivery pathways include the at least one medication delivery pathway, and wherein assigning the one or more medications for delivery to the patient via the one or more medication delivery pathways includes:
prompting a user to flush the one or more medication delivery pathways at least one of before and after delivering the one or more medications via the one or more medication delivery pathways.

5. The computer-implemented method of claim 3, wherein assigning the one or more medications for delivery to the patient via the one or more medication delivery pathways includes:
prompting a user to install the one or more medication delivery pathways at a body of the patient.

6. The computer-implemented method of claim 2, further comprising:
segmenting, with at least one processor, according to the at least one rule set, the one or more medications and the at least one medication into a plurality of medication groups based on whether the one or more medications and the at least one medication are compatible to be delivered to the patient via the same medication delivery pathway; and
determining, with at least one processor, based on the pathway data and the prescription data, according to the at least one rule set, the one or more medication delivery pathways for delivery of the plurality of medication groups to the patient based on the pathway data and the prescription data.

7. The computer-implemented method of claim 2, wherein determining whether the one or more medications are compatible to be delivered to the patient via the same medication delivery pathway as the at least one medication includes:
determining, according to the at least one rule set, based on the pathway data and the prescription data, whether the one or more medications are compatible to be delivered to the patient via a same catheter as the at least one medication.

8. The computer-implemented method of claim 7, wherein determining whether the one or more medications are compatible to be delivered to the patient via the same medication delivery pathway as the at least one medication includes:
determining, according to the at least one rule set, based on the pathway data and the prescription data, whether the one or more medications are compatible to be delivered to the patient via a same lumen of the same catheter as the at least one medication.

9. The computer-implemented method of claim 8, wherein determining whether the one or more medications are compatible to be delivered to the patient via the same medication delivery pathway as the at least one medication includes:
determining, according to the at least one rule set, based on the pathway data and the prescription data, whether the one or more medications are compatible to be delivered to the patient via a same port of the same catheter as the at least one medication.

10. The computer-implemented method of claim 1, wherein the pathway data is associated with at least one type of the at least one medication delivery pathway, and wherein determining the one or more medication delivery pathways includes:
determining, according to the at least one rule set, based on the pathway data and the prescription data, whether the one or more medications are compatible to be delivered to the patient via the one or more types of the at least one medication delivery pathway.

11. The computer-implemented method of claim 10, wherein the one or more types of the at least one medication delivery pathway include at least one of the following: a peripheral intravenous line (PIV), a peripherally inserted central catheter (PICC), a central catheter, a midline catheter, or any combination thereof.

12. The computer-implemented method of claim 1, wherein the pathway data is associated with at least one location of a body of the patient to which the at least one medication delivery pathway is configured to deliver medication, and wherein determining the one or more medication delivery pathways includes:
determining, according to the at least one rule set, based on the pathway data and the prescription data, whether the one or more medications are compatible to be delivered to the patient at the one or more locations of the body of the patient.

13. The computer-implemented method of claim 1, wherein the pathway data is associated with at least one material of the at least one medication delivery pathway, and wherein determining the one or more medication delivery pathways includes:
determining, according to the at least one rule set, based on the pathway data and the prescription data, whether the one or more medications are compatible to be delivered to the patient via a pathway including the one or more materials.

14. The computer-implemented method of claim 13, wherein determining whether the one or more medications are compatible to be delivered to the patient via the at least one medication delivery pathway including the one or more materials includes:
determining, according to the at least one rule set, based on the pathway data and the prescription data, an amount of time the one or more medications are compatible to be delivered to the patient via the pathway including the one or more materials.

15. The computer-implemented method of claim 1, wherein the prescription data is associated with one or more delivery types of the one or more medications, one or more dosage amounts of the one or more medications, one or more dosage rates of the one or more medications, one or more time periods acceptable for delivering the one or more medications to the patient, or any combination thereof.

16. The computer-implemented method of claim 15, wherein determining the one or more medication delivery pathways includes:
determining, according to the at least one rule set, based on the pathway data and the prescription data, one or more first time periods for delivery of the one or more medications via the one or more medication delivery pathways.

17. The computer-implemented method of claim 16, wherein determining the one or more medication delivery pathways includes:
determining, according to the at least one rule set, based on the pathway data and the prescription data, one or more second time periods after the one or more first time periods for delivery of one or more second doses of the one or more medications via the one or more medication delivery pathways.

18. The computer-implemented method of claim 16, wherein the at least one rule set includes a priority of delivery for the one or more medications and the at least one medication.

19. The computer-implemented method of claim 16, wherein the at least one rule set includes a priority of delivery of for the one or more medications via a type, material, and/or a location of medication delivery pathway.

20. The computer-implemented method of claim 1, wherein the at least one other medication was delivered or is being delivered via the at least one medication delivery pathway, wherein the pathway data is received from at least one sensor coupled to the at least one medication delivery pathway, and wherein the pathway data is associated with the at least one other medication delivered or being delivered via the at least one medication delivery pathway.

21. A system comprising:
one or more processors programmed and/or configured to:
obtain pathway data associated with at least one medication delivery pathway for delivering medication to a patient, wherein the at least one medication delivery pathway includes at least one of the following: at least one catheter, at least one lumen, at least one port, or any combination thereof;
obtain prescription data associated with (i) one or more medications prescribed for delivery to the patient and (ii) at least one other medication delivered, being delivered, or prescribed to be delivered to the patient;
determine according to at least one rule set, based on the pathway data and the prescription data, one or more medication delivery pathways for delivery of the one or more medications to the patient; and
assign the one or more medications for delivery to the patient via the one or more medication delivery pathways.

22. A computer program product comprising at least one non-transitory computer-readable medium including program instructions that, when executed by at least one processor, cause the at least one processor to:
obtain pathway data associated with at least one medication delivery pathway for delivering medication to a patient, wherein the at least one medication delivery pathway includes at least one of the following: at least one catheter, at least one lumen, at least one port, or any combination thereof;
obtain prescription data associated with (i) one or more medications prescribed for delivery to the patient and (ii) at least one other medication delivered, being delivered, or prescribed to be delivered to the patient;
determine according to at least one rule set, based on the pathway data and the prescription data, one or more medication delivery pathways for delivery of the one or more medications to the patient; and
assign the one or more medications for delivery to the patient via the one or more medication delivery pathways.
